(19) United States
(12) Patent Application Publication
(10) Pub. No.: US 2009/0105558 A1
(43) Pub. Date: Apr. 23, 2009

(54) Portable autonomous multi-sensory intervention device

(75) Inventors: Cheryl Riley-Doucet, Rochester Hills, MI (US); Debatosh Debnath, Rochester, MI (US); Imad H. Elhajj, Mansourieh (LB)

Correspondence Address: Ian C. McLeod;IAN C. McLEOD, P.C., 2190 Commons Parkway, Okemos, MI, 48864, US

(73) Assignee: Oakland University, Rochester, MI (US)

(21) Appl. No.: 12/287,840

(22) Filed: Oct. 14, 2008

Related U.S. Application Data
(60) Provisional application No. 60/999,163, filed on Oct. 16, 2007.

(51) Int. Cl. A61M 21/02; A61B 5/00

(52) U.S. Cl. 600/301; 600/28; 600/27

(57) ABSTRACT

A portable sensory intervention system for a patient in need thereof is provided, which comprises:(a) a soft device to be held by the patient comprising one or more stimulation units which are: (i) a speaker inside the device coupled to means for generating a sound; (ii) a light source visible outside the device for generating a soothing color; or (iii) an aroma generator, wherein the one or more of the stimulation units are coupled to a controller which is operably coupled to a wireless receiver means; and (b) a sensing unit adapted to be mounted on the patient and which detects patient agitation by electrically measuring physiological signals and wherein the sensing unit communicates with the soft device to provide instructions to the soft device to operate the one or more stimulation units in the soft device.

### CROSS-REFERENCE TO RELATED APPLICATIONS

Priority is claimed to Provisional Application No. 60/999,163, filed Oct. 16, 2007, the entire disclosure of which is herein incorporated by reference.

### STATEMENT REGARDING FEDERALLY SPONSORED RESEARCH OR DEVELOPMENT

This work was supported by a grant from the National Science Foundation (NSF)/National Institute of Health (NIH)—Grant No. 0609152; and Oakland University Provost Research Development Fund. The U.S. Government has certain rights to this invention.

### BACKGROUND OF THE INVENTION

(1) Field of the Invention

The present disclosure relates to a Portable Automated Multi-sensory Intervention Device (PAMID). The PAMID is an automated system that quantifies agitation by measuring physiological signals such as changes in heart rate, body temperature, and electro-dermal response. The PAMID further provides multi-sensory stimuli—such as music, aromatherapy, and light stimulus through colorful fiber optic lighting that reduces agitation in a Patient with Dementia (PWD). In a particular form, it resembles a soft and white stuffed animal toy like a whale which contains a multi-sensory stimulation unit. A sensing unit is designed to detect physiological responses of agitation in subjects accompanying the device. The sensing unit is wirelessly connected to the stimulation unit and to a computer that monitors physiological signals.

(2) Description of Related Art

The rapidly growing aged population brings new challenges for the current healthcare system, since it is this population that are at highest risk for developing Alzheimer's disease (AD). AD is a devastating and costly illness characterized by agitation and negative behavioral symptoms (NBS) in approximately 54% of affected patients. (See e.g., U. S. Census Bureau, “65=in the United States”, Jun. 12, 2006.) The care of elders with AD and related disorders such as agitation, costs approximately 80 to 100 billion dollars annually, which creates further burden on our presently strained healthcare delivery system. (See e.g., Rosenblatt, A., The art of managing dementia in the elderly, Cleveland Clinica Journal of Medicine, Vol. 72, No. 3, pp S3-12, 2005.) In order to control costs, provide optimal patient care, and prevent the burnout of professional and family caregivers caring for patients with dementia (PWD), efficient methods of detecting and managing agitation must be implemented to aid caregivers in their efforts.

Until recently, the use of neuroleptic drugs was the primary treatment for agitated behavior in a patient with dementia (PWD). However, results from recent studies have indicated that these medications have a limited efficacy in controlling agitation and have actually hastened the deterioration of patients' cognitive abilities. Consequently, clinical researchers are exploring alternative treatment options for managing agitation in PWD. Evidence from research indicates that complementary and alternative modalities utilizing primarily multi-sensory environments (MSE) such as music, aromatherapy, and visual stimulation are effective in relieving agitation. Typically, MSE's are set up in a single room away from a patient's room. Results from studies investigating the use of such rooms in long-term care facilities show that a common drawback is the amount of time spent by the staff members transporting a patient to and from the multi-sensory room. Another identified limitation of this intervention includes the activity of transporting the agitated patient to the multi-sensory room which may cause further confusion and distress to the patient. Furthermore, current technology used to create the multi-sensory experiences is very low-tech and requires manual setup and management on the part of caregivers. Early detection of agitation in persons with Alzheimer's disease is important in helping avoid negative sequelae associated with behavioral problems that often result from undetected agitation. Currently, detection of agitation is largely subjective and determined by caregiver observation. A need still exists for technology that can monitor and detect agitation in a PWD patient without the aid of continuous staff intervention and/or observation.

Some products currently on the market that measure physiological symptoms of agitation or stress such as; heart rate, ESR or body temperature, include the SENSWEAR armband by BODY-MEDIA, the LIVENET, and MEDNOTE. These products measure either one or more of the above mentioned physiological parameters. However, these products do not provide multi-stimulation intended to mitigate agitation or stress response. Current products providing multi-stimulation are generally low-tech and cumbersome. They typically require personnel to set up and transport patients to multi-sensory rooms. Currently, there is no technology on the market that combines the autonomous detection of agitation while providing a multi-sensory experience for a PWD patient and is also portable.

A need still exists for technology operable for detecting physiological parameters of stress or agitation along with an intervention for mitigating agitation response through a multi-sensory experience. Current technologies are prohibitively cumbersome due to large cost and/or lack of technical sophistication.

### OBJECTS

It is an object of the present invention to provide a portable device for stimulating the senses of patients. These and other objects will become increasingly apparent by reference to the following description.

### SUMMARY OF THE INVENTION

The present invention provides a portable sensory intervention system for a patient in need thereof, which comprises: (a) a soft device to be held by the patient comprising one or more stimulation units which are: (i) a speaker inside the device coupled to means for generating a sound; (ii) a light source visible outside the device for generating a soothing color; or (iii) an aroma generator, wherein the one or more of the stimulation units are coupled to a controller which is operably coupled to a wireless receiver means; and (b) a sensing unit adapted to be mounted on the patient and which detects patient agitation by electrically measuring physiological signals and wherein the sensing unit communicates with the soft device to provide instructions to the soft device to operate the one or more stimulation units in the soft device. In an exemplary embodiment, the soft device is shaped as a plush stuffed animal such as a whale. In a particular embodiment, each of the soft device and the sensing unit comprises a wireless communication device adapted to allow for wireless communication between the soft device and the sensing unit. In yet another embodiment, the system comprises a monitor station adapted to receive data from the sensing unit. The monitor station comprises a wireless communication device for communicating wirelessly with at least one of the soft device or the sensing unit. The monitor station instructs the soft device to activate at least one of the stimulation units when a predetermined threshold has been reached indicating agitation. Typically, the soft device comprises each of the sound, light, and aroma stimulation units. The means for generating a sound can be a CD player and preferably is an MP3 player.

The present disclosure provides for a sensing unit comprising a chest strap for mounting at least a heart rate monitor. In a particular embodiment, the sensing unit further comprises (i) a microcontroller mounted on the chest strap coupled to a wireless communication device; (ii) a temperature sensor for measuring temperature changes of the patient coupled to the microcontroller; and (iii) electrodermal skin response sensors for measuring change in the conductivity of the patient's skin coupled to the microcontroller.

The present disclosure provides for a portable sensory intervention device for a patient in need thereof, which comprises a soft device to be held by the patient comprising one or more stimulation units which are: (i) a speaker inside the device coupled to means for generating a sound; (ii) a light source visible outside the device for generating a soothing color; and (iii) an aroma generator. The one or more of the stimulation units are coupled to a controller which is operably coupled to a wireless receiver means for activating the stimulation units. In a particular embodiment, the soft device is in wireless communication with a sensing unit mounted on a patient for measuring physiological parameters of the patient. The sensing unit is operable to transmit a signal to the soft device to activate the stimulation units once a predetermined threshold has been reached. The soft device can be a plush stuffed animal comprising all three of the sound, light, and aroma stimulation units all mounted within the plush stuffed animal. In a particular embodiment, the animal is a whale and the stimulation units are not visible by the patient.

The present disclosure provides for a patient monitoring unit comprising: (a) a strap adapted to be cinched around the chest of the patient adjacent to the heart having a body contact side and an outside; (b) a controller mounted on the outside of the strap; (c) a wireless transmitter operably connected to the controller; (d) a temperature sensor operably connected to the controller; (e) a galvanic sensor adapted to be mounted on the skin of the patient operably connected to the controller; (f) a heart rate monitor operably mounted on the body contact side of the strap and connected to the controller; and (g) a power source for operating the monitoring unit.

The present disclosure further provides for a method of treating an agitated state of a patient in need of the treatment, which comprises: (a) providing the system of Claim 1 with the sensing unit mounted on the patient; (b) detecting agitation of the patient with the sensing unit; and (c) transmitting a signal to the soft device to activate the one or more of the stimulation units so as to calm the agitated state of the patient. The agitated state can be a result of a disease of the brain such as Alzheimer's. In a particular embodiment, the method further comprises the step of transmitting the data from the sensing unit to a monitoring station, wherein the monitoring station is in wireless communication with the soft device and transmits instructions to the soft device to activate the stimulation units when a predetermined threshold is reached.

### BRIEF DESCRIPTION OF THE DRAWINGS

FIG. 1 illustrates an exemplary system according to the present invention in use with a patient in a wheel chair.

FIG. 2 is a perspective view of a sensing unit 100.

FIG. 3 is a front view of a whale 10 as a soft toy.

FIG. 4 is a plain view of a whale 10 as a soft toy.

FIG. 5 is a perspective view showing an exemplary whale with specific components.

FIG. 6 illustrates a block diagram of an exemplary sensing unit.

FIG. 7 illustrates Block Diagram of the MSSU

FIG. 8 illustrates Block Diagram of Monitor Station

FIG. 9 illustrates a schematic for the resistance of the Thermistor.

FIG. 10 illustrates a schematic for the resistance of the GSR.

FIG. 11 illustrates a schematic of an exemplary BLUETOOTH device.

FIG. 12 illustrates a schematic for an exemplary NPN transistor switch.

FIG. 13 illustrates an exemplary startup screen of a monitor station according to the present invention.

FIG. 14 illustrates an exemplary screen shot of the trigger tab associated with the monitor station.

FIG. 15 illustrates an exemplary screen shot of the response tab associated with the monitor station.

FIG. 16A-16C are block diagrams illustrating logic for the sensing unit main and real time interrupt.

FIGS. 17A-17G illustrate block diagram schematics for the monitor station.

### DESCRIPTION OF PREFERRED EMBODIMENTS

All patents, patent applications, government publications, government regulations, and literature references cited in this specification are hereby incorporated herein by reference in their entirety. In case of conflict, the present description, including definitions, will control.

The present disclosure provides for a portable automated multi-sensory intervention device (PAMID) sensing unit. A PAMID is operable to monitor and detect agitation in an individual characterized as a patient with dementia (PWD) without the aid of continuous staff intervention. The present disclosure provides for an exemplary PAMID that is operable to automatically detect the onset of agitation in a PWD and administer stimuli that will mitigate agitation and negative behavioral symptoms (NBS) that result from undetected agitation. The present disclosure provides for PAMID systems intended to assist nurses and care providers in managing PWD and agitation thereby improving patient quality life. A further aspect of the present disclosure is the application of a specific type of non-pharmacological intervention that has been shown to mitigate agitated behaviors in PWD.

The majority of criteria designed for the measurement of agitation are largely subjective and derived from caregiver observation of specific behaviors. The present disclosure provides for a system operable of monitoring objective physiological parameters for establishing the criteria for measuring agitation in dementia. Agitation can be described as being comprised of both symptoms of physical distress and more complicated observable behaviors. (See e.g., Culter, N R., Sramek, J J., Understanding Alzheimer's Disease: For general readers a guide to understanding a devastating illness that affects a significant segment of the elderly population. Pp. 65, 82, 93, University Press of Mississippi, 1996.) The clinical index for physical distress has been associated with changes in physiological responses similar to responses found in various states of anxiety. Physiological responses include increased heart rate and body temperature, diaphoresis (resulting in increased skin conductivity), increased respiratory rate, and increased blood pressure. In an exemplary embodiment, the physiological parameters for measuring agitation in dementia were chosen to be the physiological changes seen in heart rate, body temperature, and electro-dermal skin response (EDR) which is the widely accepted method of measuring the electrical resistance of the skin during times of emotional distress.

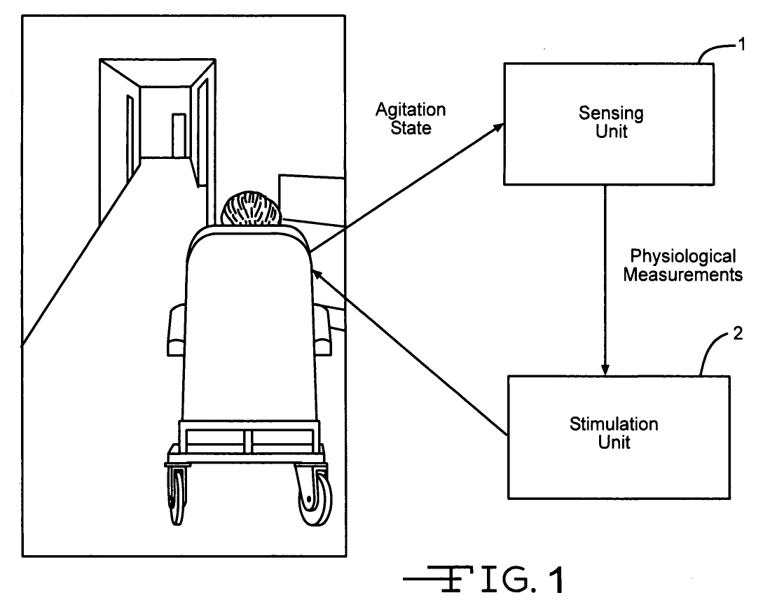

As shown in FIG. 1, an exemplary system according to the present disclosure comprises: a sensing unit 1 wherein the sensing unit measures certain physiological conditions of a patient and detects agitation through the analysis of physiological signals. Preferably, the measurements and/or detection are measured in real time. The system further comprises a stimulation unit 2 wherein once agitation is detected by sensing unit 1, stimulation unit 2 autonomously and adaptively administers appropriate sensory stimuli intended to calm the patient, i.e., relieve the agitation. Examples of sensory stimuli that would be delivered to calm the patient are fiber-optic lights, music, and aromatherapy.

In an exemplary embodiment, sensors included a POLAR exercise heart rate monitor, a 1,000 ohm platinum resistance temperature detector (RTD), and EDR electrodes that wrap around an individual's fingers. The POLAR heart rate monitor measures changes in the patient's heart rate associated with agitation. The POLAR heart rate monitor was chosen because it could easily interface with the system shown in FIG. 1, in comparison to other heart rate monitors. Moreover, the signal from the monitor is easy to analyze and it is comfortable to wear. The RTD sensor detects a patient's variations in skin temperature associated with agitation. The RTD sensor was chosen for it's small size and the ability to easily interpret the signal from the sensor using the Calendar-Van Dusen equation. These electrodes for EDR monitoring are suitable because of the inexpensiveness of the individual electrodes and their ability to attach to the patient. Some EDR sensors require that the patient's fingers be placed in or on a sensing device and do not actually attach to the body.

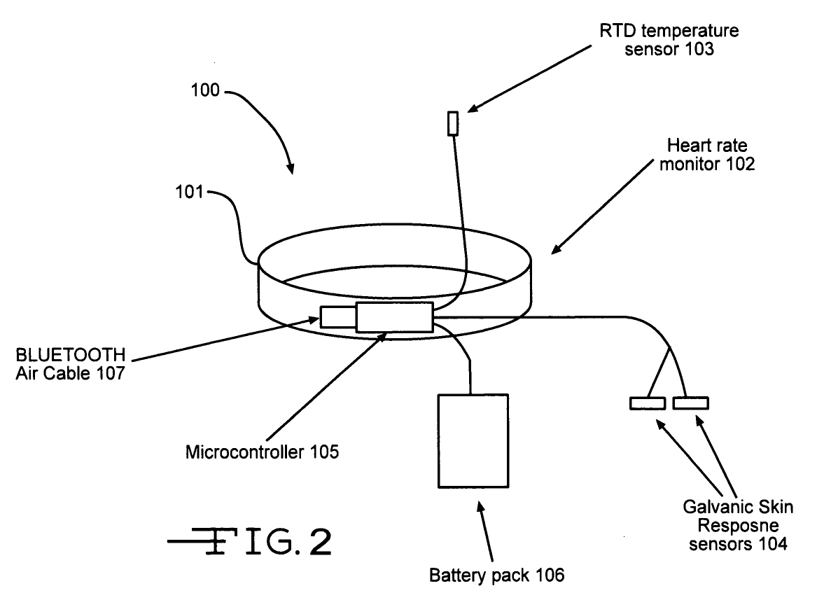

FIG. 2 illustrates an exemplary sensor acquisition system (Sensing unit) 100. Sensing unit 100 is constructed to be portable and comprises a heart rate monitor 102, skin temperature sensor 103, and galvanic skin response (GSR) sensors 104. A microcontroller 105 attaches to the heart rate monitor chest strap 101. Battery pack 106 is coupled to microcontroller 105 and its location can be moved. Resistance Temperature Detector (RTD) sensor 103 can be placed almost anywhere on the body. GSR sensors 104 must be placed around two fingers The preferred unit has the heart rate monitor 102 in the strap 101 so as to substantially be an integral unit. A BLUETOOTH device 107 is provided to wirelessly communicate with a PAMID stimulation unit as described below.

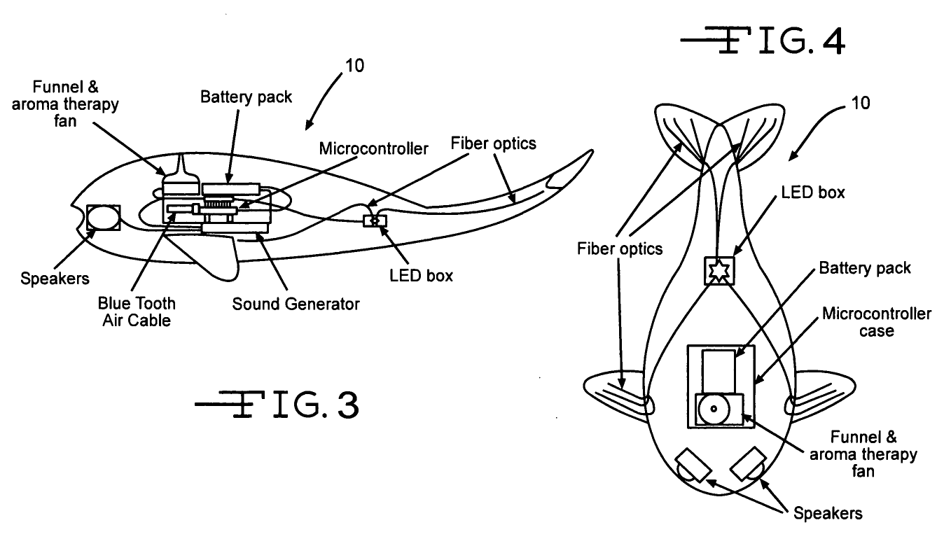

An exemplary system according to the present disclosure comprises a PAMID stimulation unit 10 as shown in FIGS. 3-5. For calming agitation, creating a multi-sensory environment in which the patient can be stimulated is a primary alternative therapy. A variety of different sensory stimulants have been used in building multi-sensory environments (MSE). Music that has been integrated into an individual's life and is based on personal preference has emerged as a dominant sensory stimulating intervention for agitation. In a particular embodiment, a portable CD player for playing the individualized music selection of the PWD is provided in the stimulation unit. The CD player is easy to use, is able to play upside down (in case the patient moves the stimuli administration device around) and has anti-skip capabilities. In a further embodiment, music is provided through an MP3 player positioned in the stimulation unit.

Aromatherapy is a known sensory stimulant that helps reduce agitation in dementia patients. Inhalation of essential oils has been found to reduce symptoms, especially restlessness, and can be used for promoting sleep, increasing alertness, reducing anxiety, as well as having positive effects on the physical condition. Lavender oil and lemon balm have been reported to be the preferred essential oil scents used to decrease agitation in dementia patients. For administering aromatherapy, an AURA CACIA pocket fan diffuser is provided in stimulation unit 10 and is desirable for its compact size and heatless diffusion method.

Light therapy is also effective and is integrated in multi-sensory stimulation for dementia patients. Light therapy has an extensive range, encompassing bright light therapy, dawn-dusk simulations, and ambient light alteration. Existing methods do not designate specific treatment guidelines. Moreover, little information favoring one method over the other is available. However, the use of fiber optic lighting is a prominent component of SNOEZELEN multi-sensory rooms. Preliminary field tests with in-home multi-sensory rooms demonstrated that, qualitatively, patients with severe cognitive impairment favored the use of colorful fiber optic lighting. Fiber optic strands and colorful LED lighting add this stimulus to the stimulation unit of the present disclosure.

In a particular embodiment, the interface between the system and more specifically the sensors to the patient, safety, comfort, transparency, and portability were taken into consideration in construction and design. The sensors shown in FIG. 2 were attached to the heart rate monitor strap 101 where the microcontroller 105 is embedded. Strap 101 is adjustable and is worn under the clothes of the individual. To make the whole system less intimidating (reduce wires) and more transparent to the patient, wireless communication between the sensing unit 100 and the stimulation unit 10 was used via BLUETOOTH device 107. FIG. 2 illustrates an exemplary sensing unit 100 associated with a PAMID system of the present disclosure.

In an exemplary embodiment, stimulation unit 10 is packaged in a soft and plush stuffed toy, typically resembling an animal. In a particular embodiment, that animal is a plush whale as shown in FIGS. 2-3. A study conducted by Nakajima et al. found that animal shaped toys could be used as a therapeutic tool for dementia patients. (See e.g., Nakajima, A. Nakamura, K., Yonemitsu, S., Oikawa, D., Ito, A., Higashi, Y., Fujimoto, T., Nambu, M., Tamura. T. Animal-shaped toys as therapeutic tools for patients with severe dementia. Proceedings of the 23rd Annual EMBS International Conference. Istanbul, Turkey, pp. 3796-8, 2001.) A plush whale is suitable because it defines an ambiguous shape, can have a light color and its association with water may produce a calming feeling. It also may be less likely to produce hallucinations as compared to other shapes/animals. Additionally, the shape of the toy allowed enough room for all of the internal components to be discreetly placed. Another advantage of this packaging choice is that the stimulation unit is portable.

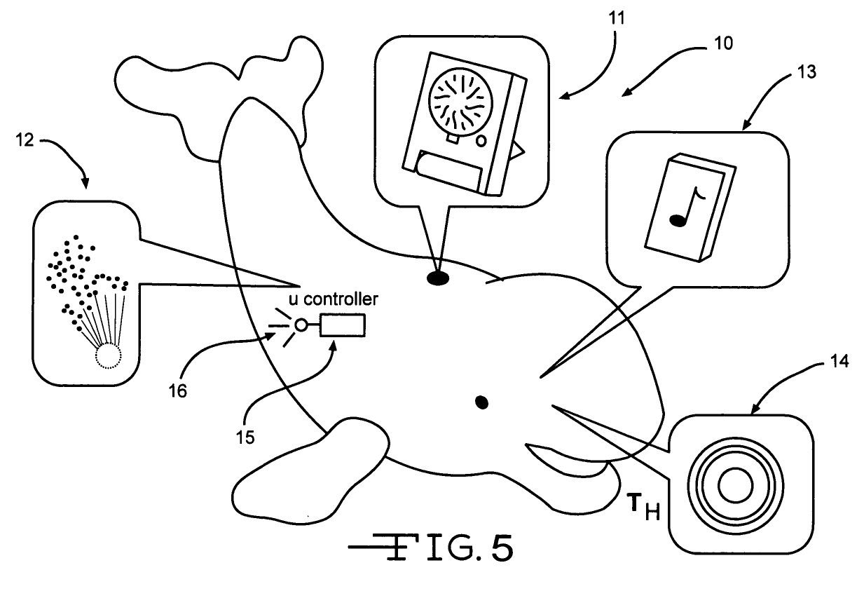

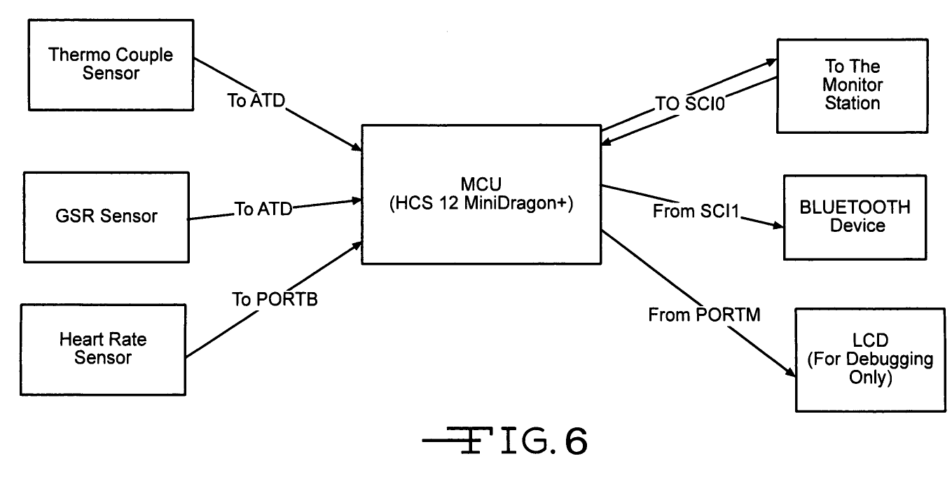

FIGS. 3 and 4 illustrate the placement of the different stimuli components in the internal body of the small whale 10. FIGS. 3 and 4 show features of the stimulation unit in more detail: Accordingly, stimulation unit 10 is made operable to play music, administer aromatherapy, and distribute lights from within. Microcontroller case 15 contains most of the stimuli components. Fiber optic cables can be removed from an LED box 12. This allows nurses to remove all electronics for replacement and cleaning when desired or necessary. FIG. 4 illustrates a plain view of the whale 10 and FIG. 3 is a front view of whale 10. FIG. 5 is a perspective view of a plush whale 10 as a soft device with an aroma generator 11, a light generator 12, a sound generator or music player 13, and speaker 14. A controller 15 with a wireless device or antenna 16 is adapted to receive a signal from the sensory unit 100 mounted on the patient.

A particular aspect of the present disclosure includes stimulation control. The system is programmed to respond to quantitatively monitored and measured agitation occurring while reducing the number of false-positives and false-negatives. The following exemplary criteria for activation of the stimulus unit to administer stimuli were defined: (a) the stimulation unit will be activated when the sensors placed on the patient detect a sharp change or reach a specific threshold of a predetermined parameter; (b) a sharp change can be defined in a particular embodiment as any combination of two of the following: (i) an increase of five (5) beats per minute detected by the heart rate monitor; (ii) an increase of one degree Fahrenheit over a five (5) minute span; and/or (iii) five (5) consecutive recessive measurements taken in EDR. The threshold is defined to indicate a significant increase from a baseline measurement. The baseline measurement is taken as an average of measurements at the start of a particular measuring process in order to be able to individualize the system to a particular patient.

In an exemplary pilot study, a significant increase was defined as an overall increase in heart rate of fifteen (15) beats per minute or an increase in temperature of 1.5 degrees Fahrenheit without relationship to time. The standard for EDR measurement will remain the same. These provisions were in place to avoid failure of the device to recognize gradual changes in the patient's response. Should the patient have reached their threshold, then the sharp change standard would be lowered so as to fully agitate. This adaptive quality individualized the system to the response of each patient. The multi-sensory stimulation device will activate for a twenty minute period or until the patient's vital signs return to normal. The twenty minute limit is in place to avoid over-stimulating the patient. See Table 1 for exemplary testing protocols.

TABLE 1
Testing activities

| Name | Purpose | Variables | Measuring Tools |
|---|---|---|---|
| Sensing unit | Technical Laboratory and | Heart rate | Polar exercise heart |
| | human subject testing to | Body temperature | rate monitor |
| | assess the adequacy of | EDR | 1,000 ohm platinum |
| | the sensing unit to monitor | | resistance |
| | the physiological | | temperature detector |
| | parameters of agitation. | | (RTD) |
| | | | Electrodes that |
| | | | wrap around the |
| | | | fingers for |
| | | | monitoring EDR |
| Stimulation Unit | Technical Laboratory and | Activation of fiber | Observation |
| | human subject testing to | optic lights, | checklist |
| | assess if; a) the multi- | aromatherapy, | Computer time |
| | sensory stimuli | compact disc music | recordings |
| | administered by the | and speakers. | Satisfaction |
| | stimulation unit are | | questionnaire |
| | triggered by changes in | | |
| | measurements of | | |
| | physiological parameters, | | |
| | b) the type and number of | | |
| | stimuli administered needs | | |
| | modification. | | |
| Control: | Testing with human | threshold values of | Computer |
| | subjects to a) assess the | heart rate, body | recordings without |
| | preliminary control | temperature and | relationship to time. |
| | scheme, and b) evaluate | EDR needed to | |
| | threshold levels to reduce | activate PAMID | |
| | false positives and/or false | stimulation unit. | |
| | negatives | | |
| Interfacing and | Assessment of the physical | subject satisfaction | Satisfaction |
| Packaging | appearance and esthetics | | questionnaire |
| | of PAMID | | |
| Subject Profile | Fulfill subject eligibility | Age, race, gender, | Demographic |
| | criteria | education level | Questionnaire |
| | | Color vision | Ishihara's Color |
| | | deficiency | Blind Test |
| | | Individual trait | Trait Anxiety |
| | | anxiety | Inventory |

The following examples are provided to further illustrate particular aspects of the present disclosure.

### EXAMPLES

A pilot study was performed to test the functionality of PAMID within a laboratory setting with healthy human subjects. As a result of these tests and assessments, the design was modified and the prototype adjusted accordingly. The following areas were tested and modified as needed.

PAMID Sensing unit: Based on the testing outcomes (technical lab and human subjects) the adequacy of the physiological parameters being monitored in detecting agitation was accessed and changes were made accordingly.

PAMID Stimulation Unit: Based on the testing outcomes the stimuli being administered triggered by changes in measurements of physiological parameters were accessed. In addition, based on subject feedback, the type and number of stimuli used can be modified.

PAMID Control System: Testing outcomes with human subjects provided data for modification of the preliminary control scheme. These tests also allowed the investigators to evaluate the sharp change and threshold values used in the control scheme and tuned them appropriately.

Interfacing and Packaging of PAMID: The physical appearance and esthetics of PAMID was modified and improved based on feedback from the human subjects. Furthermore, the acceptability of this device for use with patients and families use was evaluated based on participant feedback.

Portable Automated Multi-sensory Intervention Device (PAMID): An exemplary system according to the present disclosure is intended to aid nursing staff with methods for detecting the onset of agitation and managing agitation in older adults with dementia. The PAMID was developed by the School of Nursing and the Computer Science and Engineering Department at Oakland University, Michigan. In an exemplary embodiment, PAMID is an automated system that quantifies agitation by measuring physiological signals such as change in heart rate, body temperature, and electro-dermal response (EDR), and provides multi-sensory stimuli that has been shown to reduce agitation in persons with AD. Stimuli include music, aromatherapy, and light stimulus through colorful fiber optic lighting. In an exemplary embodiment, it resembles a soft white stuffed whale, which contains a multi-sensory stimulation unit. A sensing unit that is designed to detect physiological responses of agitation in subjects accompanies this device. The device is wirelessly connected to a microcontroller which is connected to the sensors that measure physiological signals. The sensors are also wirelessly connected to a computer that monitors physiological signals.

Prototype Design and Implementation: A prototype, as shown in FIG. 1, was designed around two major functional requirements:

1. Sensing unit: The device in real-time must objectively measure the state of a patient and detect agitation through the analysis of physiological signals; and

2. Stimulation Unit: Once agitation is detected, the device must then autonomously and adaptively administer appropriate sensory stimuli to calm the patient. Examples of sensory stimuli that would be delivered to calm the patient are fiber-optic lights, music, and aromatherapy.

Non-functional requirements for the prototype included: 1. Safety-safe for the patient and caregiver; 2. Non-intimidating to the patient; 3. Portable; 4. Low cost; 5. Easy to use by the caregiver; and 6. Customizable according to the patient's preferences.

To satisfy these functional and non-functional requirements, parameters for detecting and measuring agitation were first established and then the appropriate stimuli to be administered were set. Once these were chosen the prototype interface to the patient and its packaging was designed.

Exemplary products that have been developed by researchers which could have been used to monitor the physiological response that accompanies agitation or stress include the SENSWEAR armband by BODY-MEDIA, the LIVENET, and MEDNOTE. As previously described with respect to FIG. 2, for purposes of cost effectiveness in construction of the prototype, the POLAR exercise heart rate monitor, a 1,000 ohm platinum RTD, and electrodes that wrap around the fingers for monitoring ESR were chosen. The PAMID stimulation unit 10 as shown with respect to FIGS. 3-5 was constructed for calming agitation and creating a multi-sensory environment in which the patient can be stimulated as the primary alternative therapy.

### Research Design and Methods

A study was done that used a cross sectional quantitative study design to evaluate the overall functionality of the PAMID on healthy subjects. Since the purpose of the investigation focused on preliminary laboratory and human subject testing, healthy subjects are considered by the investigators to be the most appropriate sample group.

### Sample and Setting

Sample Size: A convenience sample of 100 undergraduate and graduate students from Oakland University were asked to participate in this study. The sample size of this study was selected to provide at least 80% power to detect what Cohen defined as medium sized effects. (See e.g., Cohen, J. Statistical power analysis for the behavioral sciences 2nd ed. Hillsdale, N.J.: Lawrence Erlbaum Associates, 1988.) Cohen defined medium sized effects as those that are visible to the naked eye, and therefore clinically important. In the case of a Pearson correlation a medium sized effect is a correlation of 0.3.

Power analysis: Power analysis conducted using PASS software, indicated that a sample of 84 subjects is sufficient to provide 80% power to detect a Pearson correlation of 0.3 with two-tailed alpha of 0.05. (See e.g., Hintze, J. L. PASS 2005 User's Guide. Kaysville, Utah: Number Cruncher Statistical Software, 2005.) The proposed sample size of 100 allowed this many subjects to be available for data analysis even if 15% are lost for some reason such as equipment failure. Power will be similar for the tests of Cohen's kappa, and higher for comparison of means (89%) and proportions responding under stress versus relaxation. Thus power will be sufficient for all planned analysis.

Sample Selection and Screening Procedures: Volunteer participants who were 18 years and older, attending Oakland University, and speak and read the English language were recruited through flyers advertising. These flyers were handed out in classrooms, placed on bulletin boards in various locations at Oakland University, and advertised in the Oakland University Press. Minority groups represent approximately 14.7% of the student population at this university (See e.g., Oakland University Student Profile, accessed from the University website), therefore it was expected that the sample for this study would include an equivalent percentage of minorities. Informed consent was obtained following the human subjects guidelines. Testing took place in a designated laboratory in one of the buildings on the university campus. As an incentive, an Oakland University “SPIRITCA$H” card of 25 dollars was given to each participant.

Screening for acceptable participants was a two-step process. The first step consisted of asking volunteers who initially responded to the advertisement for this study if they were known to be color-blind. The subjects who stated they were not color blind were asked if they would like to participate in the study and asked to read and sign a consent form. Subjects who expressed willingness to participate in the study by signing the consent form were then given a color blind test called the Ishihara Color Test (See e.g., Ishihara, S. Ishihara's test for Color Blindness, Kanehara Shuppan Co, 1973) as the second step in the screening process of the study. The Ishihara Color Test is a test specifically developed to detect color vision deficiencies in individuals. It was used in the study to screen subjects for color blindness. The Ishihara Color Test is highly reliable and is widely used to detect color-blindness. In this test there are 38 color plates, each of which contains many small dots of various sizes that are spread randomly. The dots have slightly different colors and are separated from each other by a small amount of white spaces. A number is written on the plate by using some of the dots which has a different color from the other dots. A person with color blindness is unable to read the numbers on most of the plates correctly. Usually no more than four plates are required in determining if a subject is deficient in detecting colors correctly.

It should be noted that STROOP Color-Word Interference Test which was used as a testing intervention to induce anxiety symptoms, requires non-color-blind subjects, therefore only participants who test negative for color blindness were eligible to participate in that study. The subjects who were positive to the color blind test were advised to consult their primary care physician and given an information card about colorblindness. The subjects who were negative to the Ishihara Color test were included in the rest of the study.

Stress-Inducing Intervention: A computer simulation of the STROOP Color-Word Interference Test was used to induce physiological changes in heart rate, body temperature, and EDR that are similar to those seen in times of agitation. (See e.g., Zhai, J. & Barreto, A. Stress Recognition Using Non-invasive Technology, Florida Artificial Intelligence Research Society Conference, 2006.) The STROOP Color-Word Interference Test (See e.g., Stroop, J. R. Interference in serial verbal reactions. Journal of Experimental Psychology, 18: 643-461, 1935) in its classical version, has been widely used as a psychological or cognitive stressor that can safely induce controlled limited stress in subjects. This is because the test elicits emotional responses that ultimately increase physiological (especially autonomic) reactivity. (See e.g., Renaud, P., Blondin, J. P. The stress of Stroop performance: physiological and emotional responses to color-word interference, task pacing and pacing speed. International Journal of Psychophysiology, 27: 87-97, 1997.) This has been commonly termed the “STROOP effect”. The STROOP effect capitalizes on a cognitive mechanism called inhibition. This mechanism is involved when the subject is attempting to stop a response and say or do something else. This is done by showing subjects color names written with ink of a different color and asking the subject to name the ink color, not read the color name written. For example, writing the word “red” in green and asking the subject to name the color used. The difficulty is in the fact that the tendency is to read the word faster than stating the color used. Therefore, in order to successfully carry out the test we have to inhibit out tendency to read the word. In this study, a computer-based interactive version of the STROOP test designed by Zhai & Barreto was used as a stress stimulus in the controlled laboratory environment. Previous research has indicated that by adding task pacing to the STROOP test, physiological responses intensify. Therefore in this study, each subject had 3 seconds to respond to each trial. If the subject is unable to choose and answer within 3 seconds, the screen will automatically change to the next trial.

### Data Collection:

Measurement Tools: Table 2 provides an overview of the measurement tools used to collect data for this study.

TABLE 2
Measurement tools

| | | Reliability/ | |
|---|---|---|---|
| Construct | Measure | Validity | Source |
| Individual | 20 item Trait- | R = .96 | Spielberger, |
| Trait- Anxiety | STAI | | 1983 |
| Individual | 6 item STAI- | r = .82 | Marteau & Bekker, |
| State- Anxiety | Trait Anxiety | | 1992 |
| | Inventory | | |
| Appraisal | Satisfaction | Content validity | Investigator |
| of PAMID | Questionnaire | made by panel | designed |
| | | experts | |

Trait scale—State-Trait Anxiety Inventory (T-STAI): The trait-anxiety scale is one of two subscales of the full form STAI developed by Spielberger to measure anxiety in adults. (See e.g., Spielberger, C. D. Manual For The State-Trait Anxiety Inventory STAI (Form Y). Palo Alto, Calif.: Consulting Psychology Press, 1983. Referred to as “Spielberger”) It consists of a 20 item linear analogue scale that specifically measures the more general and long-standing quality of “trait-anxiety” within each individual. The STAI is one of the most frequently used measures of anxiety in applied psychology research and has been shown to be a reliable and sensitive measure of anxiety. The T-Anxiety scale asks the respondents how they feel “generally”. Participants are asked to respond to each item on a four-point likert scale, indicating the frequency with which each strategy is used. It has acceptable reliability (r=0.96) and normative data is reported for age groups high school students, college students, 19-39 years old, 40-49 years old, and 50-69 years old with good reliability and validity.

Six item State-Trait Anxiety Inventory (STAI-6): This instrument is a 6 item linear analogue tool, developed by Marteau & Bekker (See e.g. Marteau, T. M. & Bekker, H. The development of a six item short form of the state scale of the Spielberger State-trait Anxiety Inventory (STAI). British Journal of Clinical Psychology, 31, 301-305, 1992,) to measure state anxiety in adults. It is a short version of the STAI developed by Spielberger (See e.g., Spielberger, C. D. Manual For The State-Trait Anxiety Inventory STAI (Form Y). Palo Alto, Calif.: Consulting Psychology Press, 1983) designed to measure the temporary condition of “state anxiety” when an individual is exposed to an anxiety-producing situation. It has acceptable reliability (r=0.82) and has been found to produce scores similar to the full State scale of the STAI across subject groups with normal and raised levels of anxiety. The STAI-6 is divided into three items that evaluate “anxiety present” and three items that evaluate “anxiety absent”. Spielberger states that equal numbers of “anxiety present” and “anxiety absent” items constitute a more stable measure. Questions asked respondent to rate how they feel “right now”, at this moment immediately following a stress producing event. Two of the three “anxiety absent” items are those identified by Spielberger to be particularly sensitive to low stressors and all three “anxiety present” items are those reported to be sensitive to high stressors. When compared to the full form State-STAI, the STAi-6 offers a briefer more acceptable scale for subjects. This is likely to maximize response rates, and minimize the number of missed items and response errors, thus improving the validity and generalizability of any findings.

Satisfaction Questionnaire: This short questionnaire was developed by the investigators to obtain demographic information and measure subjects' satisfaction with the comfort level of the PAMID chest monitoring system, the physical appearance of PAMID and the sensory stimuli that is administered from PAMID. Subjects are also asked to identify their age, gender, ethnic background, and highest level of education. Following this section, there were five items that use a 5-point numerical rating scale to rate subjects' satisfaction levels with PAMID; 5=very satisfied, and 1=not at all satisfied. A section was added at the end of the questionnaire for subjects to provide qualitative comments. Face and content validity were made by a panel of experts.

### Procedure

The subjects who agreed to participate in the study by signing the consent form and were negative for color blindness were asked to complete a demographic questionnaire. They were then asked to sit at a table where they completed the Trait-STAI scale. The STAI-Trait-Anxiety questionnaire was used as a covariant measure when assessing the degree of anxiety the subject experienced during the STROOP test situation. The Trait Anxiety measure indicated a subject's general anxiety level, which is an important consideration since this varied widely from person to person and impacted their response. After they completed the Trait-STAI, their pulse, body temperature by a digital thermometer and EDR were taken by a research assistant to get manual baseline data. The subjects were then instructed on how to put on the POLAR exercise strap that holds the PAMID monitoring system. Once subjects correctly put on the PAMID chest strap, data from the three sensors within the PAMID monitoring system were displayed in real-time on a computer monitor at station away from the subject. At this point, the subject's pulse, temperature, and EDR were taken again by a research assistant and these measurements were compared with those on the computer monitor to, 1) ensure the correctness of the values obtained from the sensors, 2) ensure correct position of PAMID sensing unit, and 3) establish baseline values using an average of the measurement values.

Subjects were randomly assigned to two groups. In group 1, subjects were presented with approximately 30 still, emotionally-neutral pictures as a 5 minute preliminary period of relaxation, prior to playing the STROOP test. In group 2, subjects were given a 5 minute rest period after they had finished playing the STROOP test. Comparisons between each group's PAMID recordings of physiologic measures were used to validate the sensitivity of the PAMID to respond to changes in physiological measures of anxiety.

While subjects were playing the STROOP test, four sets of data were recorded from the computer display at two minute intervals. The STROOP test was administered for 8 minutes. At the same time, research assistants observed and recorded the subjects' physical reaction and the exact time that the PAMID sensory unit was activated. The exact time of PAMID's activation was also recorded on the computer display.

Once subjects completed the STROOP test, they were asked to complete the STAI-6 and the satisfaction questionnaire. Subjects who were randomly assigned to group 1 were asked to remove their chest strap. Subjects who were randomly assigned to group 2 were allowed to rest for a period of five minutes before removing their chest strap. The STAI was used as a validation of state anxiety in subjects produced by the STROOP test. Results from STAI were compared with the physiological measures of anxiety in subjects recorded on PAMID. The whole procedure took approximately 30 minutes to complete. After completing the STROOP test and the required questionnaires each participant was given a $25 dollar Oakland University Spirit Ca$h card and thanked for their time and help in the investigation. Confidentiality was ensured through identification numbers and data was kept in locked file containers.

### Data Analysis

Preliminary to analysis, descriptive statistics (e.g. Means, median, standard deviations, ranges, skewness, kurtosis, number of missing cases, etc.) were computed on all variables in order to describe the sample and to determine the manner in which individual variables should be treated. Item analysis, including coefficient alpha and item to total correlations, was computed for all scales to determine the quality of the measures and integrity of the data before proceeding. In addition, checks for uni-variant and multi-variant outliers were made. Missing data was dealt with using the EM (expectation-maximization) method where appropriate. All statistical tests were at the conventional 2-tail 0.05 alpha level unless otherwise indicated. See table 3 for description of analysis of specific aims.

TABLE 3
Analysis of Specific Aims

| AIM | Research Question | Statistical Tests |
|---|---|---|
| 1. Accuracy &sensitivity | What is the difference between the | Crosstabulation |
| of PAMID to measure | recordings of heart rate, body | analyses will be done |
| heart rate, EDR &body | temperature and EDR taken from | to determine the |
| temperature during | the PAMID sensing unit and | relationship between |
| STROOP test. | manual measurement methods? | measurements of |
| | | physiological |
| | | parameters of anxiety |
| | | taken by manual |
| | | methods and those |
| | | from the PAMID |
| | | sensing unit. |
| | | Direct measure and |
| | | test of agreement |
| | | using Cohen's |
| | | weighted Kappa |
| | | statistic. Weighting |
| | | will be selected to |
| | | punish the score more |
| | | for bigger deviations |
| | | between the two |
| | | measures. |
| 2. Reliability of PAMID to | How often does the PAMID | Frequencies |
| follow control method of | stimulation unit activate when the | distributions (Yes - |
| administering multi-stimuli | sensing unit detects an increase in | No) of observation |
| and stop administering | heart rate of 15 beats/min., and | checklists and |
| stimuli when physiological | increase in body temperature of 1.5 | computer recordings. |
| threshold measurements | degrees Fahrenheit without | Agreement assessed |
| have been reached. | relationship to time? | by the weighted |
| | How often does the PAMID | Kappa statistic. |
| | stimulation unit stop administering | |
| | multi-stimuli when the sensing unit | |
| | recordings reach below threshold | |
| | levels for heart rate, body | |
| | temperature and EDR? | |
| 3. To determine the | What are the levels of satisfaction | Frequency |
| acceptability and comfort | with regards to comfort of the | distributions and |
| level of PAMID to | PAMID chest monitoring system, | mean to summarize |
| research subjects. | the physical appearance of PAMID | survey results. |
| | and the sensory stimuli that is | |
| | administered from PAMID? | |

### Results

PAMID measured heart rate, skin temperature, and electro-dermal response by using a set of sensors attached to a subject. Heart rate and skin temperature that PAMID measured were compared with manually measured values for each subject. It was observed that the difference between heart rate measured by the two methods was only one to three beats per minute. PAMID measured skin temperature with less than 0.5 degree Fahrenheit accuracy.

PAMID detected the following changes in physiological parameters during four minutes of non-congruent STROOP test:
Average change in heart rate: 19.05 bits/min.
Percent change in heart rate: 23.37%
Average change in skin temperature: 1.48° F.
Percent change in skin temperature: 2.51%
Average change in electro-dermal response: 96.89
Percent change in electro-dermal response: 16.32%

Out of 100 subjects, only temperature data for two subjects were not captured properly because of malfunctioned sensors. Only for one subject, the heart rate monitor was unable to collect any data.

Each subject reported on whether or not PAMID was pleasing to look at and whether the sensing unit was comfortable to wear. Overall participants indicated that they were satisfied with the PAMID device. On average on a scale from 1 to 3, 3 being very pleasing and 1 not at all pleasing, they reported a satisfaction with the appearance of PAMID (M=2.7), and on a scale from 1 to 3, 1 being not at all comfortable and 3 being very comfortable, rated the sensing unit to be quite comfortable to wear, (M=2.6). Only 1% of the subjects indicated they did not know if PAMID was pleasing to look at and if it was comfortable to wear.

Importance of this Research and Future Directions.

The impact of the proposed study and the device is two fold. First the PAMID technology will enhance care planning strategies for patients with Alzheimer's disease and optimize their treatment through providing real-time customized adaptive care. Second, by assisting healthcare providers in early detection of agitation in PWAD, quality of care will be improved. Another impact of the proposed research is its value to researchers in studying the therapeutic effect of multi-sensory stimulation (MSS) and its benefits in relieving agitation in dementia patients. This device provides researchers with an abundance of data which is difficult to acquire otherwise.

The following equipment was used in testing the exemplary PAMID embodiments:
2× Aircable Wireless Devices
plush whale
Aromatic diffuser
1× 12V battery (2 were available)
4× LED arrays (each array had a switching transistor included)
Heart rate monitor and chest strap

Some equipment that was used at first but then substituted latter includes:
RTS—The original RTS (resistance temperature sensor) had a linear output which made it easy to read but had large range and therefore there was very small resistance change for small changes of temperature.
GSR—there were two types of GSR, one that would mount on fingers and one that would mount on skin. The skin GSR consisted of about 24 single use pads. The skin GSR pads were used during the initial testing but soon were replaced with electro gel which was effective.

Equipment that was provided and then not used include:

The 3× microcontrollers from freescale were provided. While these MCU's were small and easy to use they had limitations for example they had one SCI port and our sensing unit needed to communicate with both MSSU and the monitoring station at once. This was not possible by using these MCUs.

The CD player was replaced by a MP3 player.

The CD player protective case was made of wood and to heavy to be included within the whale. A cardboard box was used instead.

The original speakers were not amplified meaning that they drew power from the device that was also supplying the music. It is very hard to get any volume from MP3 players or any small devices without amplification. A pair of speakers that had a sound amplifier attached was used instead.

The initial testing was broken into four steps: (1) be able to gather data from all sensors using a MCU; (2)—evaluate data and check for agitation by evaluating the threshold conditions; (3) make the decision to turn on PAMID based on data gathered by the sensors; and (4) turn on PAMID wirelessly.

Once testing began, it became evident that more steps had to be added. It was decided to add a monitoring station, a GUI interface that would allow the user to monitor all vital signs and the state of PAMID. This would also serve as a debugger for the project. All of the aspects of PAMID were made programmable through windows interface so that the nursing counterparts would not find it difficult to use.

Inside MSSU (i.e., the whale), a MP3 player with an advanced amplified sound system was installed and an array of LEDs with different colors individually controlled directly by the MCU and indirectly by the user, was implemented. A power circuit with voltage regulators and protection diodes was designed and implemented making PAMID portable and independent. The BLUETOOTH device was connected to its counterpart and the MCU establishing the connection between the MSSU and the sensing unit.

On the sensing unit the heart rate, GSR, RTS were calibrated. The original RTS was replaced for one that had faster response and more changes for the desired range of operation. The original GSR was also replaced for a more robust one. Both GSR and RTS were mounted on the chest strap and care was takes so they didn't bother the user. Aspects of PAMID were made programmable and the heart beat, temperature and skin resistance were updated instantly on the GUI. Data captured was automatically written into a file (every second) for latter analysis. The latter versions of PAMID calibrate themselves for every individual user.

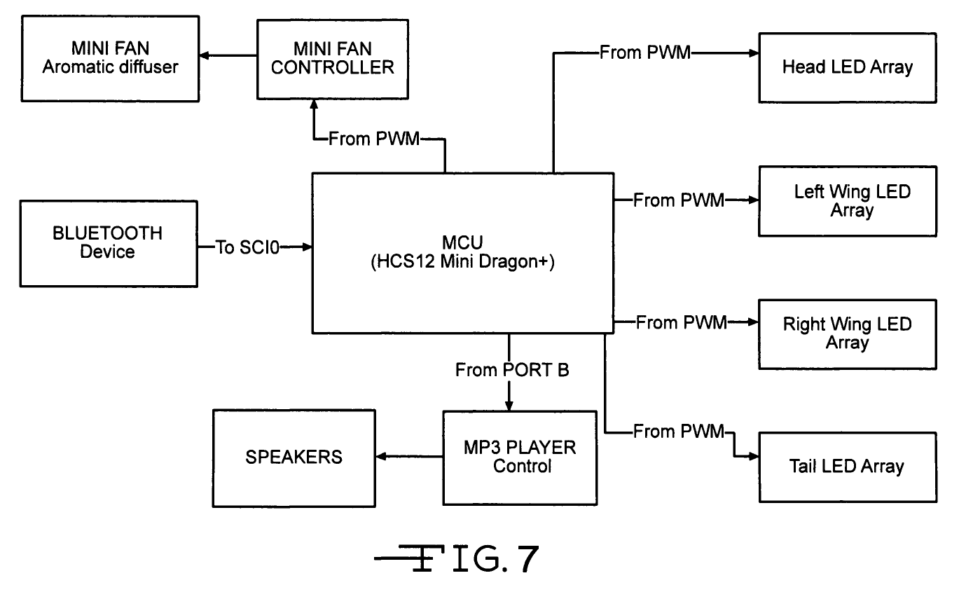

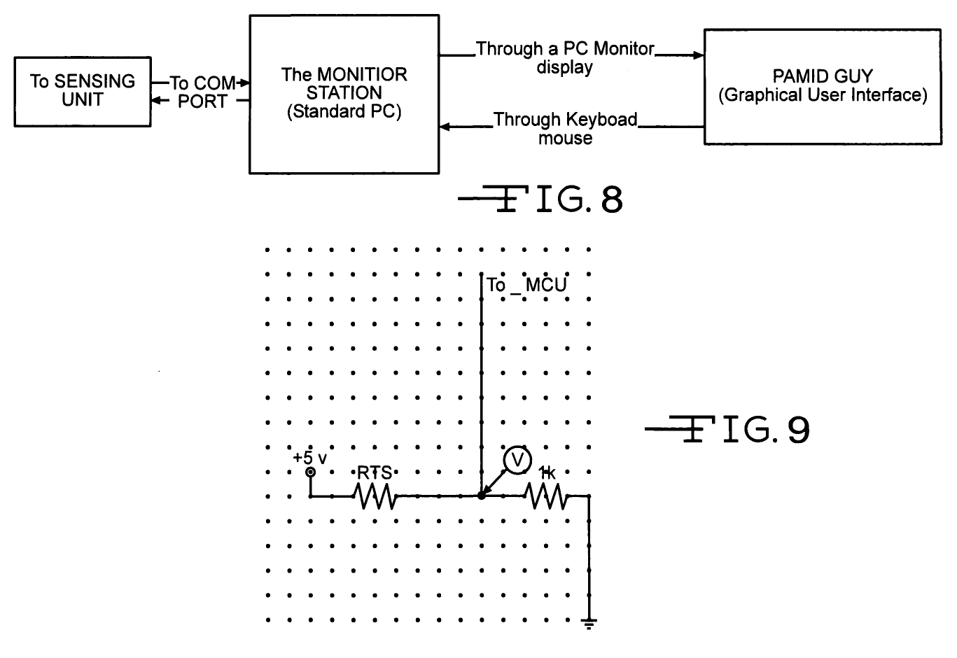

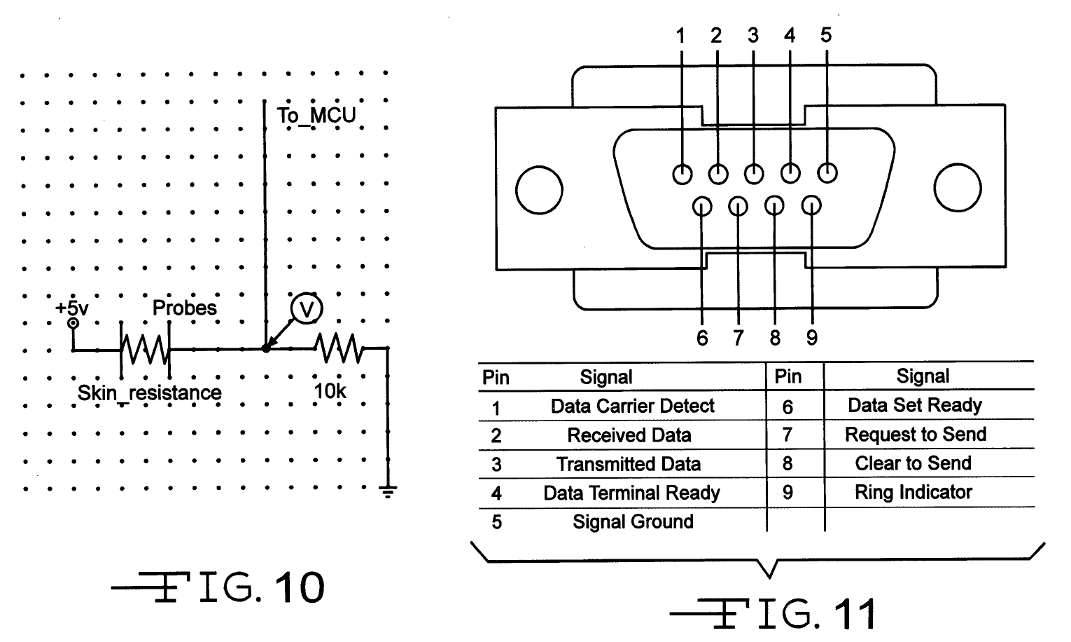

FIG. 6 illustrates a block diagram of a sensing unit according to the present disclosure. The sensing unit will communicate with a monitor but it will only send commands to MSSU. The MCU will read data from the sensors, manipulate the data and send it through the serial communication to the monitor station as shown in FIG. 7. Some data is sent to the LCD screen which is only for debugging purposes. When the decisions have been made to turn on the PAMID, data will go through the BLUETOOTH device to the MSSU and activate it. The MSSU is connected to four arrays of LED, a standard mini aromatic defusing unit and a fan, and an MP3 player. It will accept data from sensing unit and execute the proper instructions. FIG. 8 illustrates a Block Diagram of Monitor Station.

### Sensing Unit

In an exemplary embodiment, the sensing unit comprises the following components: a HCS12 microcontroller from FREESCALE, a POLAR heart rate monitor and a wireless receiver, a resistance based temperature sensor, a galvanic skin response sensor, a BLUETOOTH wireless device from AIRCABLE, and a standard 9v battery. The sensing unit can be distinguished into two parts: (a) the station which includes the microcontroller, a BLUETOOTH device, a battery and a breadboard where some simple circuit is being implemented (while testing the station can sit anywhere close the subject being tested); and (b) the chest strap is the remote device. It must be mounted on the subject's chest and later connected to the station. The chest strap typically is comprised of the following sensors: RTS (Resistance based temperature sensor), GSR (Galvanic skin response sensor) and the heart rate monitor itself.

### MCU (MicroController Unit)

The HCS12 MINIDRAGON+ is a standard 16 bit microcontroller. This particular one was chosen because of its compact size and its many onboard devices. Science Sensing unit has to communicate with both the MSSU and Monitor Station at the same time a microcontroller with two serial communication ports was needed. The MINIDRAGON+ has two SCI (serial communication interface) ports, SCI0 and SCI1. SCI0 is used to communicate with monitor station and uses a standard baud rate of 9600. A baud rate of 9600 is considered pretty low but speed was not an issue on this application. The connection between SCI0 and monitor station is done through a standard wire but it can very easily be replaced with a BLUETOOTH device to eliminate the wires. The microcontroller continuously sends sensor data through the SCI0 and pauses for 300 ms before resending again. Data is sent eight bits at a time and that means that the largest number that can sent is 255. All data is sent in raw mode and not ASCII characters because the later was not necessary for the exemplary operation. However, 10 bit numbers were used to collect the data so two (bytes) transmissions were used to send just one number. This is done by sending the sending the 8 (MSB) most significant bits first and then sending the next 8 (LSB) least significant bits. The receiver must know the sequence in order to make sense of the data. Serial communication is a two way communication and the sensing unit also receives data through SCI0. Information entered from the user on the monitor station is transmitted to the sensing unit the same way the sensing unit transmits to the monitor station. When data arrives through the serial port SCI0 an interrupt is used to capture it and then later process it. This is necessary to ensure that no data is lost.

The second port, SCI1 is used to communicate with the MSSU. This is done wirelessly via a BLUETOOTH module mounted on the sensing unit. As in the first one the baud rate is 9600 and this port is used only to transmit, thus not receiving any data because the MSSU is not connected to any sensors.

The HCS12 also has 2 ATD (Analog to Digital) converter modules each consisting of 8 channels so 16 total ATD channels. Only two channels were used since only output analogue voltage was needed. It takes about 30 ms for the ATD module to complete one conversion. A 10 bit ATD conversion was used which combines two channels together and uses only 10 LSB out of 16 available. This gives a resolution of 1024/5 volts or about 5 mv resolution. It is desired to get high resolution so the testers do not miss important changes on the subject's vital parameters. The HCS12 does these conversions periodically at about 3 Hz (there is a 300 ms delay on the program to ensure this). This is slow in microcontroller terms but it is desired as not to overwhelm the system with data.

This microcontroller is also capable of generating periodic interrupts. The interrupts are needed to calculate time and heart rate. The heart rate sensor has a 3.3V output pulse whenever there's a heart beat. To detect this real time interrupt is used to check the port which the heart rate sensor is connected. A 3.3V logic is high in TTL (transistor logic) so no external circuitry was needed. Typically, an op-amps to scale the output to 5 volts is used. The calculation of the heart rate is done according to the following procedure: Run the real time interrupt at 10.24 ms intervals. Check to see if the PORTB BIT 0 (this is the port where heart rate sensor output is connected) is high. If it is high, then record the time and compare that with the last time. This will give the time between two beets in multiples of 10.24 ms. So if between two beats there was one sec difference, the result would be 97*10.24=1 sec. This is the time between two beats but it is inversely connected to the heart rate. Finally, convert this to BPM (beats per minute) because that is a more familiar term.

Power comes to the microcontroller throughout a 9V battery or a 7.5V adapter. It goes through a 5V 1A regulator that produces enough power for all the components of this application. Besides the MCU other devices that use power include: the BLUETOOTH device, the heart rate wireless receiver, the GSR, and the temperature sensor (RTS).

The BLUETOOTH device is second to the MCU in power consumption followed by the heart sensor wireless receiver. The RTS and GSR have high resistance values so they use very little power. Power consumption of sensing unit varies greatly on the power consumption of the individual components which also varies on the operation they are performing. On average the whole system consumes 300 mA @7.5V, approximately 2.25 W. The greatest variance to power consumption comes from the BLUETOOTH device and the MCU. The MCU will use more power when sending through the serial port and making ATD conversion. The BLUETOOTH device uses more power when sending information and when it cannot find the other BLUETOOTH device because it constantly looks for it.

### Heart Rate Sensor

The POLAR heart rate monitor is made by POLAR and the wireless receiver is made from VERNIER. In an exemplary embodiment, the device is further made of two sub devices, a) the POLAR chest strap and b) the VERNIER wireless receiver. A suitable chest strap is made by POLAR. These devices are very robust and work under a variety of conditions. The human heart expands and contracts to allow blood with low oxygen to flow to the lungs and blood with high oxygen to flow to the body and brain. The heart beat is controlled by autonomous nerves which output an electric current to signal the heart muscles to contract. The heart muscle in itself works just like any other muscle in the body. The electric signal that goes to the heart is very minor but it is enough to be captured by a very sensitive device. This is also the concept behind the POLAR chest strap. The main unit which should be put as close the heart as possible will “listen” for these pulses and once it detects one it will capture it and use its energy to create another pulse at 5 KHz and output wirelessly to the receiver. This is a great advantage because by using the energy from the heat electromagnetic pulse, the device does not need to be supplied with power. The chest strap has adjustable features and it is designed to fit on a variety of people.

The wireless receiver is the counter part of the chest strap. It is composed by a strong antenna that receives the week pulse, amplifies it and sends it the MCU. It is an active device, i.e., it has to be supplied with power. It uses 5V and it is powered from the 5V regulator. The maximum range is 110 cm. There are three significant connections that come from the receiver and they are: Ground, VCC and data. The data line will output an electric pulse every time there is a heart beat. The pulse has a magnitude of 3.3V which is not considered TTL (transistor logic) but fortunately it is regarded as a one (1) in TTL because the MCU will consider all signals over 2.5V to be a one (1) and all signals under 2.5V to be a 0. This means that this line can be used directly without having to implement external circuitry for amplifiers and transistors thus preventing onboard ATD conversion which takes time and consumes unnecessary power. Once the 3.3V pulse comes in, it takes about 100 ms for the line to stabilize to 0V. The MCU does not read any data until the line has been stabilized. An interrupt running at 10.24 ms is checks to see if this line is high and disables it until it goes low. These steps are repeated. It will also calculate the time between two consecutive beats. After the line goes high it will fall to negative voltage for a short period of time so it is very important to disable the line until it is stable again. The error in calculating the heart rate this way can be as much as 20.48 ms per beat. On a 60 beats/min heart rate this accounts for a 2% maximum error.

### RTS (Resistance Based Temperature Sensor), Thermistor

Finding the temperature of a surface can be done in two different distinct ways, active and passive temperature sensors. Active temperature sensors are comprised of a diode, which is extra sensitive to temperature, and some extra circuitry to amplify and calculate the change in voltage through the diode. These sensors are very accurate and have high resolution. The drawback is that they have a slow response. Once the temperature changes it may take two minutes to output to represent that change.

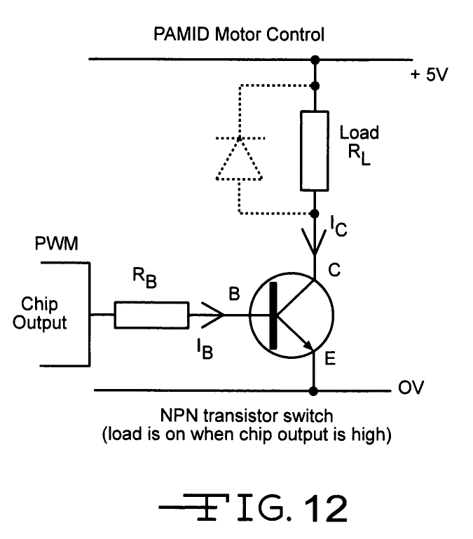

Passive temperature sensors are comprised of a resistance of some kind that changes value with the change in temperature. There are different kinds of RTS based on range of temperatures they accept and the change on resistance they offer. Regular RTS offer a linear change but cover a wide range of temperatures while offering low resolution. Thermistors on the other hand have a logarithmic change in resistance for linear change in temperature but offer greater resolution on particular ranges. In an exemplary embodiment, a Thermistor was chosen and offers large changes in resistance at ranges from 0-100° C. The sensor is mounted on the chest strap and is in direct contact with the subject's body skin. Because the temperature of the human body is not evenly distributed on all the parts of the body there may be a one-two degrees drop depending on the section being measured. This temperature drop is also different for different people and further varies with their mood, prior activities and clothing they are wearing. Regular thermometers (e.g., digital or mercury) readings have to be taken on the armpit or on the mouth where the sensor (in the digital case) or the mercury tank, are enveloped with the subjects skin. In the tests associated with the present disclosure, it would be unpractical to do this, thus only 40% of the sensor surface is in direct contact with the subject's skin. This accounts for another 1% drop on the output temperature. The objective, however, is not to measure the exact temperature but measure the change in temperature. The Thermistor is very sensitive in this regard and will pick up very small changes. Before the Thermistor is used it has to first be put in a voltage divider or a Wheatstone bridge. The bridge balances the circuit and has an output of 0V when the circuit is on normal condition and goes positive or negative depending on the change. This would not be very useful in present testing because work with negative voltages (ATD conversion takes 0-5V) was not feasible. The other option is a simpler voltage divider. The resistance of the Thermistor is around 10 kOhm at 22° C., thus simply connecting it in series with another 1 kOhm resistance as seen in FIG. 9 was done. When the resistance of the sensor gets smaller than 10 kOhm, then the value in the middle node will be closer to 5V and vice versa. An increase in voltage output can be selected with an increase in resistance or the opposite by interchanging the 5V with the ground connection. The middle node is connected to the ATD module on the MCU and measures the voltage on that node and converts it into a 10 bit integer corresponding to the voltage.

The value has to be further modified so a temperature reading can be found. Thermistors have logarithmic output. Three different temperature readings were measured: one point for low temperature (0 degree), another point for mid temperature (22 degrees) and another for high temperature (40 degrees). The Stein-Heart equation was used to describe the logarithmic behavior of the Thermistors:

1/T=A+B*(Ln R)+C*(Ln R)³

T=1/[A+B*Ln(R)+C*(Ln(R))³]

Where T is temperature in Kelvin and A, B, C are constants which were determined by the above three experiments.

### GSR

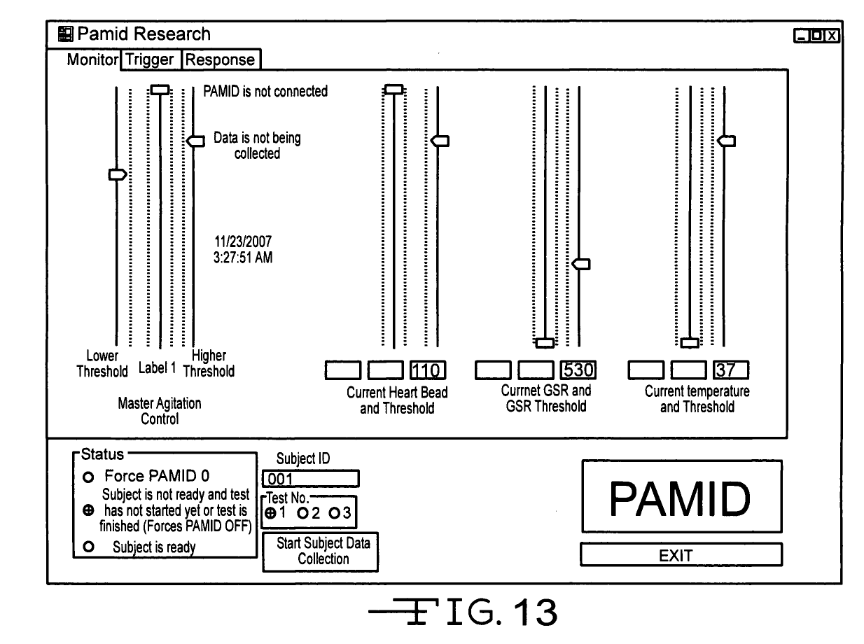

Galvanic skin response (GSR) refers to the conductivity of a persons flesh and skin. According to the present disclosure, the GSR is used to detect agitation. The concept behind GSR is based on the fact that a person's conductivity (or resistivity) changes when they are agitated. This happens because the skin will produce more sweat when a person is stressed. The GSR in an exemplary application consists of two probes that are in direct contact with the subject's skin at about 5 cm apart and have a difference in electric potential. Electric current will flow from the probe with the highest potential to the probe with the lower potential. The current flowing is directly dependent on the resistance that it encounters. If the resistance is put in a series with another resistance than a voltage divider can be created allowing for measuring a change in resistance by measuring the voltage on the middle node as seen in FIG. 10. When the two resistances are similar the voltage output is 2.5V. Otherwise it changes linearly according to the change of resistance. The output is fed into the MCU ATD and a reading is taken about three times per minute. The GSR has the fastest response of all the sensors as the change in resistance of the body will instantly change the voltage on the middle node. The GSR value has Ohm units but because it is used to detect a change in the subject's vital characteristics it is not converted to match the real skin resistance. This is also because skin resistance is very different on different individuals and also depends on the state of the person. The resistance decreases when a person gets agitated because they sweat more making it easier for current to go through. For similar reasons as those discussed on the RTS paragraph above a voltage divider is used instead of a Wheatstone bridge on this application. Since metal-skin connection is not a strong connection, most medical applications use electro gel to facilitate readings. Thus electro gel is used on the GSR probes for this application.

### BLUETOOTH Device

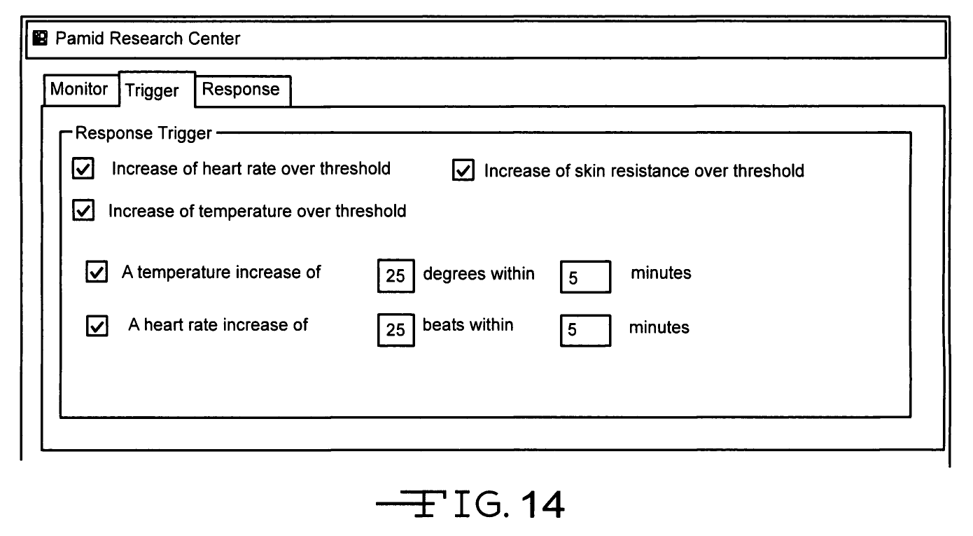

The BLUETOOTH devices used in an exemplary application are made by AIRCABLE and they come in pairs. Every device is in itself a receiver and a transmitter, however the one attached to the sensing unit will be referred to as a transmitter and the one attached to the MSSU will be referred to as a receiver. The transmitter is connected to the MCU SCI1 and operates at a 9600 baud rate. This particular device can be powered in two ways: a) using a standard adapter with a barrel connector, or b) through pin 9 as shown in FIG. 11. It accepts any voltage between 5 and 15V and consumes variable power depending on the transmission rate frequency speed and range. The BLUETOOTH device is powered on the sensing unit through the 5V source that is produced by the 5V 1A regulator on board the MCU. The pair of BLUETOOTH devices makes sure that all information coming on the TX pin goes to the Rx pin of the opposite device and vice versa. When trying to connect two MCUs together as is the case on this particular application, the RX and TX pins must be interchanged for the communication to be established. In an exemplary embodiment, this is done on the MSSU side of the connection. In an exemplary embodiment, when a BLUETOOTH is turned on it starts to look for another device. The pulsating blue LED indicates that the module has power and is looking for another device but has not found one. When another active device comes within the maximum range of operation the two recognize each other and establish a communication between each other. The blue LED stays on when a communication has been established. Pressing the pair button on both devices at the same time will synchronize these devices together making them not discoverable to other devices. This way more than a pair can be used. These devices only work if the data being sent or received is of the RS232 format. They have a range of 30 ft anywhere or 50 ft in-line of sight. Communication rate varies from 4800 bps to 112500 bps and can be changed by selecting the right combination of switches on the module itself. The module as a SUB-D 9 male connector in which pin 2 and 3 are TX/RX. The interchanging of these wires is also called a null modem configuration. In FIG. 11, the real TX signal must be connected to the RD and the RX must be connected to the TD to create the null modem.

### Multi-Sensory Stimulation Unit

The Multi-sensory Stimulation Unit (MSSU) refers to the response of the system and in an exemplary embodiment, is physically included in a plush whale. It is comprised of the following components: HCS12 microcontroller (MCU); BLUETOOTH wireless device; 24 LEDs; an MP3 player (e.g., Sansa Express); amplified speaker system (e.g., Insignia); a mini fan attached to an aroma pad; a 12V 1800 mAh rechargeable battery; and a plush whale stuffed animal.

As mentioned above the HCS12 MINIDRAGON+ was chosen for the MSSU for similar reasons it was chosen for the sensing unit. Its small size (2.2″×3.2″) makes it ideal to fit almost anywhere. Theoretically speaking the MSSU required less peripheries and components than the sensing unit since most of the logic and calculations are being implemented on the sensing unit. The MCU on this device is operable to performing the following tasks: receive information about its status and manage the LEDs, the MP3 player and control the fan. It will need to use one SCI, one PWM and seven general purpose I/O devices to archive all tasks.

General purpose I/O can be any pin in the MCU and the MINIDRAGON+ has 89 I/O pins. However most pins have double functions so care must be taken when choosing which I/O pins to use. To use a general I/O pin first it must be designed as output or input by setting the DDR (Data Direction Register). A value of one will specify the port is setup as an output and a value of 0 for the port as an input.

### PWM (Pulse Width Modulation)

PWM is used to control the speed of the fan that powers the aromatic diffuser. An I/O pin could have been used to set it on or off but this way using PWM allows control of the speed of the fan. PWM specifies the width and the period of the output signal. The duty to period ratio specifies the power output which will later be amplified and fed to the motor. If the duty cycle is 0 than the output is 0V, however as the duty increases the voltage output increases. The duty Voltage is 5V while the non duty voltage is 0V.

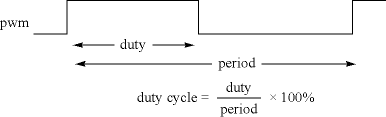

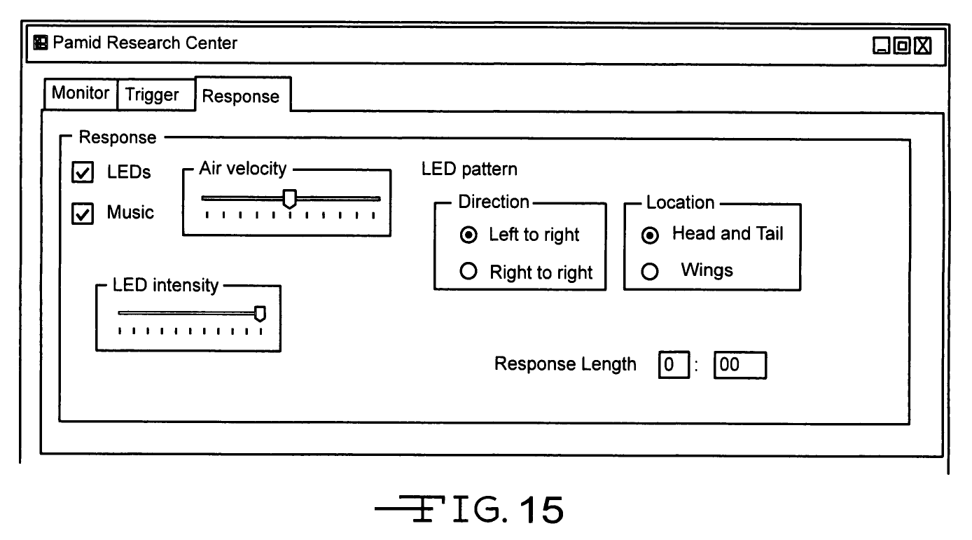

Even though the average of the output is a voltage between 0-5V, there is very little power associated with it since these pins are supplied by the MCU. In order to drive any significant load with them, they first should be isolated from the load. This is done by a transistor, MOSFET, Op-AMP, ¼ H Bridge or solid state relays. NPN transistors were used because of low cost and ease of use. FIG. 12 shows R load representing the motor and Vs=5 volts. As shown in FIG. 12, the motor is not drawing any power from the MCU because of the properties of NPN transistor in which the base B is isolated form the emitter E and collector C. A diode can be used on larger motors to prevent back EMF from damaging the transistors but our motor is very small and the diode is not necessary (Back EMF is created when the power is cut to the motor but it is still spinning because of its inertia).

### BLUETOOTH Wireless Device

The second Bluetooth device is used to finalize the connection between the sensing unit and PAMID. Since both MCUs on these devices are the same type (they both transmit on pin 2 and receive on pin 3) their input and output signals are a concern. As explained above (see the sensing unit BLUETOOTH device, null modem section) a null modem has to be implemented on one of the MCUs. The RX and the TX wires are interchanged on the MSSU to establish communication. The baud rate has to be the same as the sensing unit and the baud rate of both wireless devices for this function to work.

### LED (Light Emitting Diode)

LEDs were implemented on the interior skin of the plush whale and light-up when the response is on. There are four sections each with six LEDs connected for a total of twenty four. Once the response is on the head section LEDs turn on, than the right wing section, the tail and finally the left wing section. Each stays on for half a second and it can be adjusted to the patients need. Also the pattern can be adjusted form the monitor station. In this configuration no more than six LED are on at the same time, which limits their current consumption to 200 mA. It may be desirable to keep this to a minimum because of battery life and to protect the LEDs. LED is a diode and has minimal resistance. When connection to any power supply it will let large amounts of current go through it which will result in overheating and burnout of the diode. This has been corrected by attaching 330 Ohm resistances to all LED. The LED's also need transistors so they can be turned on or off. One transistor is needed to control one section or a total of six LEDs. The LEDs are the first response. The second response is music implemented through a MP3 player and an amplified speaker system.

### MP3 Player (e.g., SANSA EXPRESS)

The SANSA EXPRESS is a standard MP3 player with 1 GB memory and it can also be used as a storage device. Songs or music can be downloaded from any pc through the USB interface. Songs can also be organized in albums or playlists which can be played at any time.

In order to use the MP3 player with the present disclosure, some modification were made. The device has a power button that can turn it on and off. Ideally, one switch is provided for turning it on and another for turning it off. The on/off switch on the MP3 player has to be pressed for more than 2 sec for the device to turn on or off. The same switch is also being used to access the menu bar. When the order comes for this device to turn on the output goes high for 2 sec and than goes low again. The MCU keeps track of the state of this device. When the order to turn it off comes, the output goes high for another 2 sec. It is desired to sync the device with the MCU since there no clear way to check if the device is on or off. One of the drawbacks of this particular MP3 player is that it resets the volume every time it restarts to the middle level. While the volume can be increased through the amplifier, it is much better quality wise to have your device output high volume and amplify less than low volume and amplify more. This device has its own battery that will run for 18 h and is recharged through the USB port. When the device is connected to a PC through the USB port is also recharging its battery. The output of the MP3 player is a standard high quality two channel headphone driver. In order to connect it with any speaker system, its signal must be amplified first. The MP3 player also has other features not currently being used like a LCD screen, volume control, FM, AM radio capabilities and record and playback devices. In an exemplary embodiment, a record and playback device can be implemented to transmit the patient's voice to the caregiver in case he or she has an emergency.

Amplified Speaker system (e.g., INSIGNIA)

An INSIGNIA model was chosen because it was a relatively small amplified speaker system. After disassembling the speakers, the following components were obtained: two standard 8 ohm speakers, and power amplifier circuit with volume control. Total power output is 4 W but that is calculated at max volume. The physical volume decreased a lot after disassembly. The right speaker was mounted on the interior right of the whale while the left speaker on the left of the whale. The amplifier circuit was isolated to decrease any chance of short circuit and mounted on the lower tail of the whale. There is one output that goes to the speakers and one input that comes from the battery. It accepts 7.5V of DC voltage and it is sensitive in quality to voltage ranges different from 7.5V. The volume control is implemented through a potentiometer that can be adjusted depending on the need. The main battery outputs 12V so a ZENER diode is used to scale that down to 7.5V.

### Mini Aromatic Fan Diffuser

In an exemplary embodiment, this little fan is located on the top section of the whale and it is connected to a funnel and will blow air outside when activated. It is fed with power through a transitory and a battery and controlled by the MCU. It will take air from the inside of the whale making it pass through an aroma pad and propel it on the outside. All aspects of this fan are customizable including the aroma pad and fan blade size and angle. The speed that the motor runs comes through the serial connection and from the monitor station and is setup by the caregiver. By default it is at 2.5V which is a moderate speed or half of maximum. The fan is bidirectional although only one direction is needed. The fan's power doesn't come directly from the battery but through the 5V 1A regulator on board the MCU.

### Monitor Station

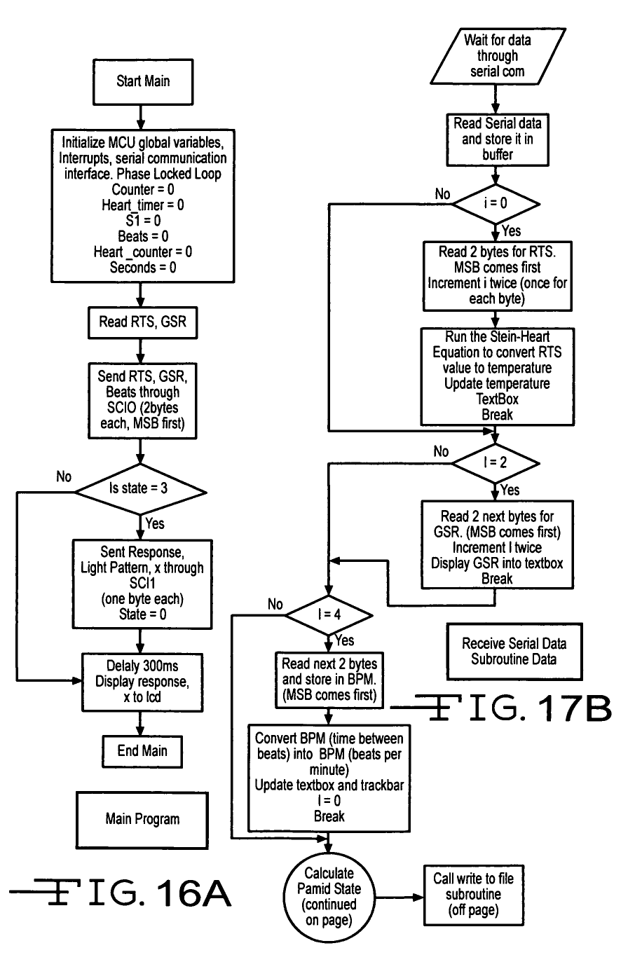

The monitor station refers to GUI (graphical user interface) software that can be accessed through a standard PC. FIG. 13 illustrates an exemplary startup screen of the monitor station according to the present disclosure. The software was written in Visual Basic and is used to change most aspects of the sensing unit and the MSSU. It can do this by communicating with the sensing unit and changing parameters (variables) such as temperature threshold, or GSR threshold or how the response of the whale should be implemented (i.e., with music and lights and aroma or just music or just lights and so on). There are three sections (classes) that make this software work, main form which is represented in FIG. 13, CRS232 class which handles the serial communications and FILEWRITER class which make sure that data coming in from the sensing unit is being saved on the disk.

### CRS232

This is a class (library) that facilitates serial communications. It will wait on the background for serial data to come to the serial port and it will capture that data and store it in a buffer. Once there is data on the buffer it will give an event (same as interrupt for MCUs) that the main program can capture and service. The buffer size is variable but doesn't need to be large because most PCs are much faster than the HCS12 and that means that they will be able to get the data before new data comes in. The class also has functions that can be called and given parameters to output to the serial port. The baud rate is being set when the main form is loaded and can be changed to match the MCU baud rate. Default baud rate is 9600 bps.

### FILEWRITER

This class has two functions and handles file I/O. It will create a file with the name of the test subject and will store data in it every second. One way of writing files includes checking to see if a file of the same name exists and choosing to overwrite or simply adding data to the end of the file. In a particular embodiment, adding data at the end of the file was chosen to reduce loss of data when human error occurs. This is done by setting the append attribute to true. The first function is called to write the header of the file which is in the following format: File for subject_test 001_{—}001.txt.

In a particular embodiment, the interface is setup such that the first column represents temperature of the subject, the second column represents skin resistance of the subject, the third column represents heart rate, and the fourth column represents time and date of the test. All the files have the suffix .txt and are in standard ASCII format, which makes them easy to read for any processor.

### Main Form

The main form is the main program that links everything together and organizes the data received from the sensing unit. It also makes complex calculations to convert the temperature from digital value representing an analogue voltage (represented by a 10 bit number) to final temperature in Celsius with 0.1° C. accuracy. It will also calculate the heart beats per minutes given time between two beats in milliseconds. It is named monitor station because an operator (usually a nurse) can monitor this information with no knowledge of any programming language and that individual can vary most parameters of the PAMID. This is done by the GUI which maps all the variables of the PAMID to textboxes or track bars that can be easily seen and visualized by the operator.

The main program revolves around receiving and converting data (from raw to ready to use) and displaying it. Every time data comes from a serial port (COM 1 in most cases) it gets stored as a buffer and creates an event which can be captured and serviced. The buffer keeps track of what was read and stored and all the data is in local variables. Some values (like GSR and Temperature) are in 10 bits so two bytes for each of these is received. Once everything is stored in local variables the counter is reset and then waits for the other set of data. Meanwhile this data needs processing and delivery to the screen and/or written to the file (if the option is checked). Since computers are generally faster than most MCUs, overflowing the buffer is not an issue.

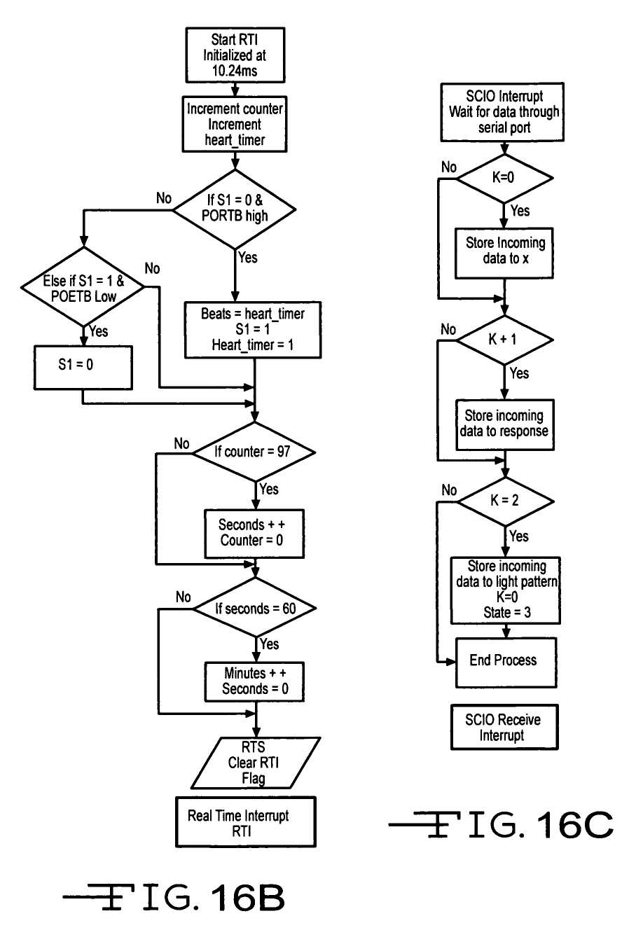

When one of the conditions for PAMID response has been reached, the program moves from state 0 (normal state) to a different state (depending on what triggered PAMID). At any state but 0, PAMID goes into a countdown at the end of which it will turn on. Different states have different countdown timers with the smallest being 5 sec and largest being 50 sec. Once PAMID is on it will go through another countdown timer which is variable but by default is set to 6 min. There are different countdown timers here as well depending how PAMID was triggered (i.e., if PAMID was triggered because values went a lot over their thresholds or increased really fast, than PAMID will stay on longer). When PAMID is on but is about to go off it will check to see what state the subject is in. If the subject is still agitated it will extend itself for another minute. It will keep extending its timer until the subject is calmer (i.e., values are below thresholds). All of these parameters are accessible through GUI and no knowledge of programming language is needed to change them. This can be done in the trigger tab of the main from as shown in FIG. 14.

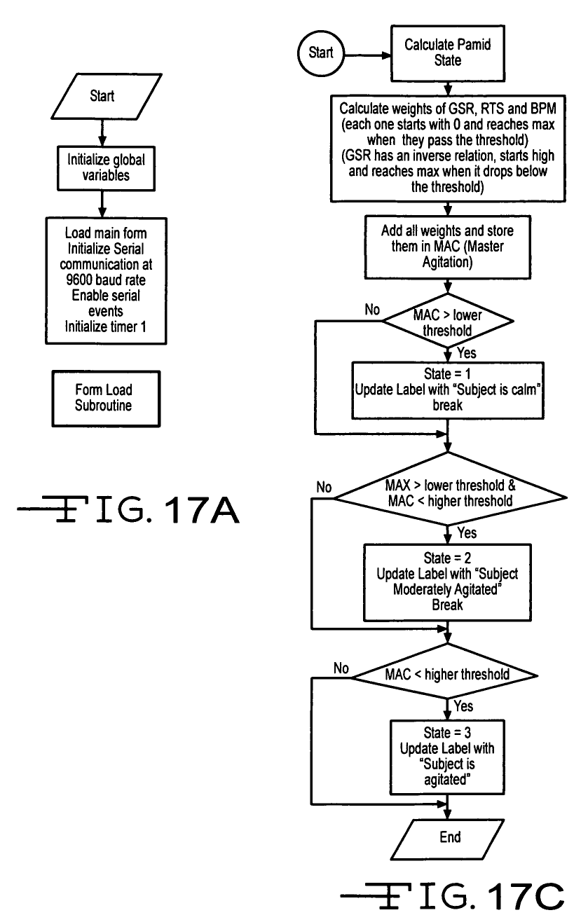

The response tab can be used to setup the response of the MSSU as can be seen below in FIG. 15. Almost every aspect of the whale can be changed in this panel and will apply to the MSSU. Data from this tab goes serially to the sensing unit and then via BLUETOOTH to the MSSU.

### Logic of PAMID

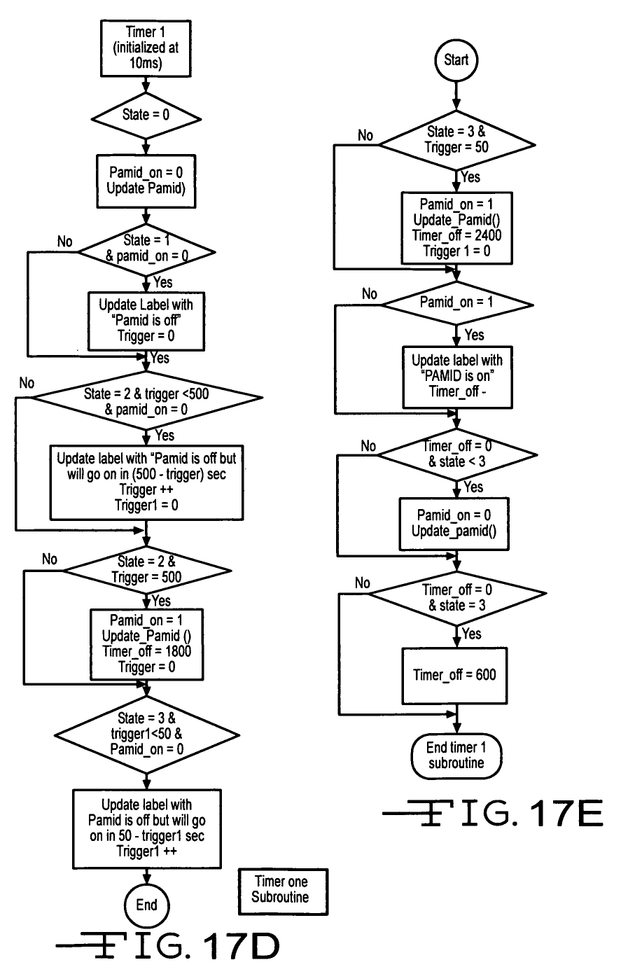

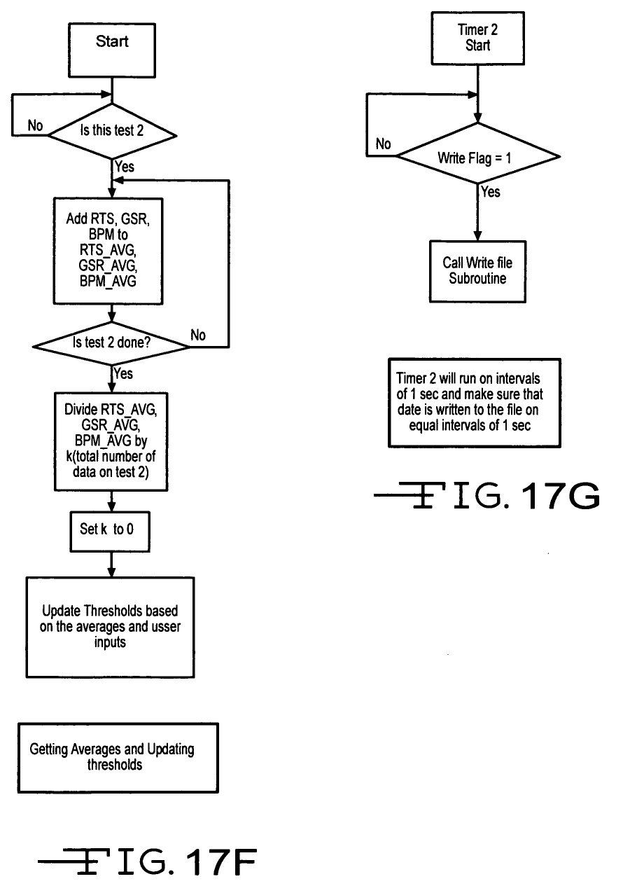

FIGS. 16A-16C are block diagrams illustrating the logic for the sensing unit main and real time interrupt. The two interrupts are on the left and the main program runs on the right. FIGS. 17A-17G illustrate block diagrams schematics for the monitoring station.

There are three devices in an exemplary application and three different computer programs that govern each of them. Each folder for each device contains a separate project that has to be compiled and downloaded to the appropriate device. The first two projects (Sensing Unit and MSSU) should be compiled and downloaded on the sensing unit and the MSSU respectively. The third project (VBPAMID) is the source code and libraries for the monitor station and should be compiled and built in any .net environment. Since MSSU and sensing unit are written on the same language and for the same board only sensing unit is described.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the Claims attached herein.

We claim:

1. A portable sensory intervention system for a patient in need thereof, which comprises:
(a) a soft device to be held by the patient comprising one or more stimulation units selected which are selected from the group consisting of: (i) a speaker inside the device coupled to means for generating a sound; (ii) a light source visible outside the device for generating a soothing color; and (iii) an aroma generator, wherein the one or more of the stimulation units are coupled to a controller which is operably coupled to a wireless receiver means; and
(b) a sensing unit adapted to be mounted on the patient and which detects patient agitation by electrically measuring physiological signals and wherein the sensing unit communicates with the soft device to provide instructions to the soft device to operate the one or more stimulation units in the soft device.

2. The system of claim 1 wherein the soft device is shaped as a plush stuffed animal.

3. The system of claim 2 wherein the animal is a whale.

4. The system of claim 1 wherein the each of the soft device and the sensing unit comprises a wireless communication device adapted to allow for wireless communication between the soft device and the sensing unit.

5. The system of claim 1 further comprising a monitor station adapted to receive data from the sensing unit.

6. The system of claim 5 wherein the monitor station comprises a wireless communication device for communicating wirelessly with at least one of a member selected from the group consisting of the soft device and the sensing unit.

7. The system of claim 5 wherein the monitor station instructs the soft device to activate at least one of the stimulation units when a predetermined threshold has been reached indicating agitation.

8. The system of claim 1 wherein the soft device comprises each of the sound, light, and aroma stimulation units.

9. The system of claim 1 wherein the means for generating a sound is a CD player.

10. The system of claim 1 wherein the means for generating a sound is a MP3 player.

11. The system of claim 1 wherein the sensing unit comprises a chest strap for mounting at least a heart rate monitor.

12. The system of claim 11 wherein the sensing unit further comprises (i) a microcontroller mounted on the chest strap coupled to a wireless communication device; (ii) a temperature sensor for measuring temperature changes of the patient coupled to the microcontroller; and (iii) electrodermal skin response sensors for measuring change in the conductivity of the patient's skin coupled to the microcontroller.

13. A portable sensory intervention device for a patient in need thereof, which comprises:
a soft device to be held by the patient comprising one or more stimulation units which are selected from the group consisting of: (i) a speaker inside the device coupled to means for generating a sound; (ii) a light source visible outside the device for generating a soothing color; and (iii) an aroma generator,
wherein the one or more of the stimulation units are coupled to a controller which is operably coupled to a wireless receiver means for activating the stimulation units.

14. The device of claim 13 wherein the soft device is in wireless communication with a sensing unit mounted on a patient for measuring physiological parameters of the patient wherein the sensing unit is operable to transmit a signal to the soft device to activate the one or more stimulation units once a predetermined threshold has been reached.

15. The device of claim 13 wherein the soft device is a plush stuffed animal comprising all three of the sound, light, and aroma stimulation units all mounted within the plush stuffed animal.

16. The device of claim 15 wherein the animal is a whale and the stimulation units are not visible by the patient.

17. The device of claim 13 wherein the means for generating a sound is an MP3 player.

18. A patient monitoring unit comprising:
(a) a strap adapted to be cinched around the chest of the patient adjacent to the heart having a body contact side and an outside;
(b) a controller mounted on the outside of the strap;
(c) a wireless transmitter operably connected to the controller;
(d) a temperature sensor operably connected to the controller;
(e) a galvanic sensor adapted to be mounted on the skin of the patient operably connected to the controller;
(f) a heart rate monitor operably mounted on the body contact side of the strap and connected to the controller; and
(g) a power source for operating the monitoring unit.

19. The monitoring unit of claim 18, wherein the wireless transmitter enables wireless communication with a soft device comprising one or more stimulation units which are: (i) a speaker inside the device coupled to means for generating a sound; (ii) a light source visible outside the device for generating a soothing color; and (iii) an aroma generator, wherein the one or more of the stimulation units are coupled to a controller which is operably coupled to a wireless receiver means for activating the stimulation units.

20. A method of treating an agitated state of a patient in need of the treatment, which comprises:
(a) providing the system of claim 1 with the sensing unit mounted on the patient;
(b) detecting agitation of the patient with the sensing unit; and
(c) transmitting a signal to the soft device to activate the one or more of the stimulation units so as to calm the agitated state of the patient.

21. The method of claim 20 wherein the agitated state is as a result of a disease of the brain.

22. The method of claim 21 wherein the disease is Alzheimer's.

23. The method of claim 20 further comprising the step of transmitting the data from the sensing unit to a monitoring station, wherein the monitoring station is in wireless communication with the soft device and transmits instructions to the soft device to activate the stimulation units when a predetermined threshold is reached.
